(19) United States
(12) Patent Application Publication
(10) Pub. No.: US 2026/0053216 A1
(43) Pub. Date: Feb. 26, 2026

(54) SENSORISED WEARABLE TEXTILE DEVICE, GARMENT INCORPORATING THE SAME AND CORRESPONDING MANUFACTURING METHOD

(71) Applicant: ACCYOURATE GROUP SPA, L’Aquila (IT)

(72) Inventors: Giuseppe ARNALDO USAI, L'Aquila (IT); Andrea GIULIANI, L'Aquila (IT); Andrea AGRUSTI, L'Aquila (IT)

(73) Assignee: ACCYOURATE GROUP SPA, L’Aquila (IT)

(21) Appl. No.: 19/102,435

(22) PCT Filed: Aug. 10, 2023
(86) PCT No.: PCT/IB2023/058118

§ 371 Date: Feb. 10, 2025

(30) Foreign Application Priority Data
Aug. 10, 2022 (IT) 102022000017160

(51) Int. Cl. A41D 13/12; A61B 5/00; A61B 5/256; A61B 5/268

(52) U.S. Cl. CPC A41D 13/1281; A61B 5/256; A61B 5/268; A61B 5/6831

(57) ABSTRACT

A band-shaped multilayer textile device includes an electrode unit cut from a screen printed sheet of an electrically conductive ink. A top equipped with such band and a manufacturing method for the band are also presented. The band textile device includes at least one electrode unit, a first textile layer, a second layer, at least one electrical terminal, a mechanical coupling device, at least one layer of adhesive material, and a closure device.

### CROSS-REFERENCE TO THE RELATED APPLICATIONS

This application is the national phase entry of International Application No. PCT/IB2023/058118, filed on Aug. 10, 2023, which is based upon and claims priority to Italian Patent Application No. 102022000017160, filed on Aug. 10, 2022, the entire contents of which are incorporated herein by reference.

### TECHNICAL FIELD

The present invention refers to a sensorized wearable textile device for detecting electrical signals for estimating the electrocardiogram and respiratory rate of a user wearing this device. The latter can be provided in a garment for greater precision in the position of the sensors and for greater ease of handling even by untrained users, e.g. staff of service companies, industries, etc. who wear the device while working.

### BACKGROUND

Garments are known for detecting electrical signals feeding known algorithms for estimating the electrocardiogram and respiratory rate. In sports, the detection of electrical signals is favored by sweat and implementation is simplified since training sessions have a relatively limited duration. However, when it is necessary to collect worker data during working hours, it is important to identify a manufacturing layout suitable for large number production, precise enough and comfortable to wear for long periods.

### SUMMARY

The scope of the present invention is to provide a sensorized textile device capable of satisfying at least in part the needs indicated above.

The scope of the present invention is achieved by means of an elastic band textile device for adherence to the body comprising:
at least one electrode assembly made by screen printing an electrically conductive polymer on a flexible substrate
the electrode unit comprising a screen-printed pad in contact with the skin of the user in use and a screen printed elongated filiform element defining electrical continuity with the pad the electrode assembly being cut from a pre-screen printed sheet with the electrically conductive polymer
a first textile layer in use on the skin side having a corresponding first opening for allowing the pad in use to contact the skin
a second layer opposite the first textile layer with respect to the electrode assembly and having a corresponding second opening for exposing a portion of the filiform element
at least one electrical terminal fixed in electrical continuity with the filiform element through the second opening and arranged on the surface of the second layer to be used connected to a control unit configured to receive and process the electrical signal of the pad
a mechanical attaching device configured to carry the electronic control unit while it is in electrical continuity with the electrical terminal
at least one layer of adhesive material to fix the first textile layer to the second layer, the electrode group being interposed, to define a band
a closure member for releasably connecting first and second end portions of the band to each other and allowing in use the band to adhere to the skin of the chest when adjusted.

A sensorized band configured in this way provides a sufficient comfort when worn for long periods through the first textile layer, being in particular of textile material the area around the electrodes. The multilayer construction is simple to manufacture and to provide large production batches and is flexible enough around the chest to have adequate comfort over long periods of use and adapts to the user's chest.

In the same way, the electrode unit obtained from a subsequently cut screen-printed sheet allows to reduce waste to a minimum, to precisely control the deposition of the ink and have precise geometries of the component and prepare the process for large production batches. Furthermore, through screen printing, it is possible to incorporate the pad and the elongated filiform conductor that carries the electrical signal to the control unit in a single element, simplifying the production process.

The use of adhesive on both layers sheathes the filiform conductor in order to make adhesion efficient and at the same time reduce the disturbances of the electrical signal along the filiform conductor.

Other advantages of the present invention are discussed in the description and referred to in the dependent claims.

### BRIEF DESCRIPTION OF THE DRAWINGS

The invention is described below on the basis of non-limiting examples illustrated by way of example in the following figures, which refer respectively to:
FIG. 1 a plan view of some electrode assemblies for a device according to the present invention;
FIG. 2 an enlarged schematic view of a section of the device in a manufacturing phase prior to gluing;
FIG. 3 an enlarged schematic view of a section of the device on the skin side; and
FIG. 4 an enlarged schematic view of a section of the device on the opposite skin side; and
FIG. 5 an enlarged perspective view of an electrode unit and a mechanical connection element for electric conduction applied thereon.

### DETAILED DESCRIPTION OF THE EMBODIMENTS

FIG. 1 shows electrode units E for a wearable textile device according to the present invention. These electrode units E are preferably made starting from a sheet screen-printed almost completely with an electrically conductive ink, e.g. a peripheral portion of the sheet is not screen printed.

On this screen-printed area several electrode units E are obtained by cutting the sheet, for example by laser cutting. Preferably, as illustrated in FIG. 1, the cutting operation includes changes in direction and/or non-straight cutting lines to obtain the shaped peripheral edge of each electrode assembly. Therefore, the conductive ink is present on the entire cut area or in any case on most of it and the conductive ink is found on the entire cut perimeter or on most of it. In this way, it is possible to obtain geometries of electrode units E to adapt to the three-dimensional configuration of the band. For example, the configuration of electrode units E illustrated in the figures is substantially one-dimensional so as to affect the bending and torsional stiffness of the band as little as possible, thus increasing comfort.

Screen printing provides a greater control of the uniformity of the thickness of the conductive ink compared to other techniques such as spraying, and is aimed at maximum reduction of the thickness of the “added” materials on the sheet, in order to cancel the perception of “foreign bodies” on the sensorised and skin-contact portions of tissue. Additional techniques for applying electrically conductive ink include immersion in a bath or the use of screen printing masks through which the ink is sprayed. The application of the ink on the sheet is such that the thickness of the ink is substantially uniform, in particular the ink has uniform thickness on the electrode units E. According to a preferred embodiment, the sheet is made of fabric, so as to increase the comfort of direct contact with the skin. Even more preferably, the fabric comprises a yarn based on polyester (PE) with additives of ceramic and elastane (EA) particles. For example, ceramic particles are oxides of one or more metals, such as zinc, platinum, titanium, aluminium and silicon. The percentages of these particles do not exceed 5% by weight and the additives can be either by incorporation into the polyester before extrusion of the yarn or by coating on the yarn. The use of these particles makes it possible to increase the electrical insulation of the elongated filiform conductor and reduce the disturbances along this conductor on the electrical signal picked up by electrode unit E, in particular in the presence of the user's sweat. However, it should be noted that sweat increases and stabilizes the conductivity of the electrically conductive polymer in contact with the skin.

According to a preferred embodiment, the conductive polymer comprises polyethylenedioxythiophene: sulphonated polystyrene, hereinafter PEDOT: PSS, and at least one of dimethylsulfoxide DMSO, ethylene glycol EG and sorbitol, preferably the former in a greater quantity by weight than the latter. Alternatively or in combination, it is also possible to functionalize the polymer through metallic nanoparticles, for example Ag, Au, Ag/AgCl. It is also possible to add a crosslinker to the polymer, for example (3-glycidyloxypropyl)trimethoxysilane (GLYMO) to improve the mechanical characteristics, and/or a surfactant, for example Triton X or p-dodecylbenzenesulfonic acid (DBSA), to improve application on the sheet by screen printing. As illustrated in FIG. 1, each electrode unit E comprises, preferably in a single body cut from the sheet, a pad P in use in contact with the user's skin and an elongated filiform element F coming out of pad P. As illustrated in the drawings, elongated filiform element F is narrower than pad P and carries the electrical signal detected by pad P, which acts as an electrode, through the screen-printed layer of electrically conductive ink which covers the entire functional surface of electrode unit E. Preferably, ink layer on pad P is not in relief with respect to that on the elongated filiform element F.

FIG. 2 schematically shows a further production step of the textile device of the present invention wherein electrode units E, e.g. at least three for the detection of signals suitable for estimating the user's electrocardiogram, are coupled, on the opposite side of the screen printed surface, with an adhesive e.g. a hot-laminate adhesive film such as a thermoplastic polyurethane (TPU) based adhesive. For example, the film has 40% elongation with a 4N load and 100% elastic recover when applied to a 10 mm tape. In order to facilitate the production method, the adhesive is a double-sided adhesive film.

Furthermore, the textile device of the present invention comprises a first textile layer S1 in use in contact with the skin and a second layer S2, preferably also textile and even more preferably of the same fabric as textile layer S1, in particular in a single body with layer S1 as illustrated in FIG. 2. Preferably, textile layer S1 is made with 50% Polyurethane fibers (Pu), 36% Polyamide fibers (Pa) and 14% Elastane fibers (Ea).

With reference to FIG. 2, a further layer of adhesive is applied to a band of fabric, preferably the same as electrode unit E.

Furthermore, in order to allow the electrical signals to be picked up and transmitted, first layer S1 has openings A1 through which pad P is used in contact with the skin. Preferably, pad P is superimposed on first layer S1 towards the skin and the remaining part of the electrode unit E, through the corresponding opening A1, is separated from the skin through the first layer S1. In particular, pad P, as shown in FIG. 2, is on first layer S1 out of the corresponding opening Al along a direction of elongation of elongated filiform conductor F. This ensures good contact of pad P with the skin. As shown in FIG. 2, openings A1 are slots having at least a maximum dimension smaller than a maximum dimension of corresponding pad P. In this way, waste material is reduced. For example, a larger side of opening Al is smaller than a larger side of pad P.

Similarly, second openings A2 are made on the second layer S2 to expose the surface of an electrical terminal T electrically connected to the elongated filiform conductor F. Preferably, the electrical terminal T is made of an electrically conductive metal and is arranged in relief with respect to the second layer S2. For example, the electrical terminal T is crimped to the filiform electrical conductor F.

According to a preferred embodiment of the present invention, the textile device is further provided with a boss B of a thermally conductive material e.g. metallic having a first surface arranged in use in contact with the skin and a second surface uncovered on the second layer, such that the second surface is representative, after a suitable heat transmission time, of the temperature of the skin.

Preferably, terminals T and boss B are close together so that an electronic control unit, releasably carried on the textile device, is simultaneously connected to exchange electrical and temperature signals with terminals T and boss B. For example, at least one between terminals T and boss B acts both as a signal transmission element and as a mechanical attaching element for the electronic control unit. For example, boss B is a male or female element of a snap button that releasably connects the control unit to band D. Furthermore, as shown in FIG. 3, terminals T and boss B are arranged in a non-axisymmetric figure so that the control unit has only one fixing angular position on band D.

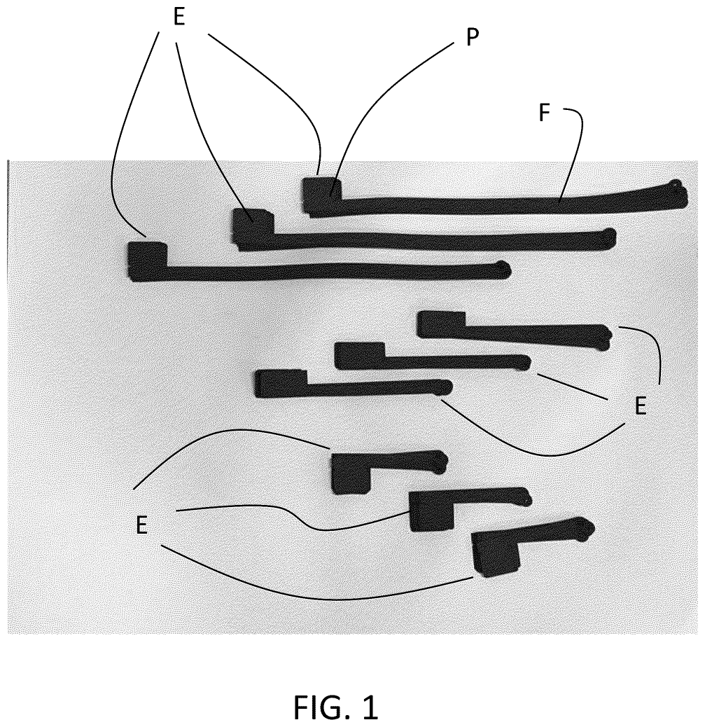

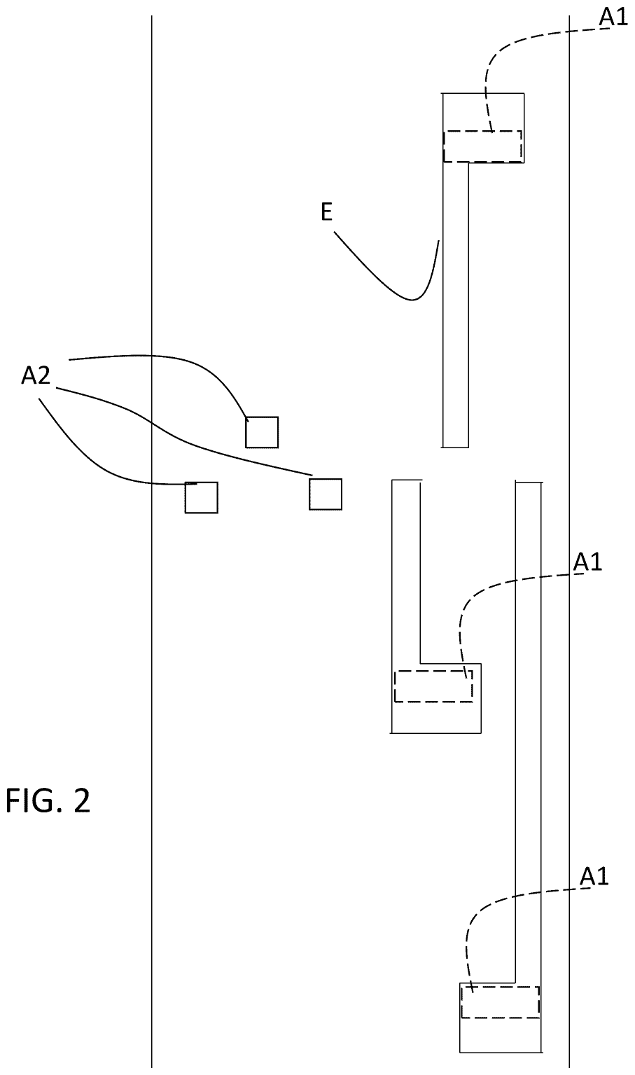

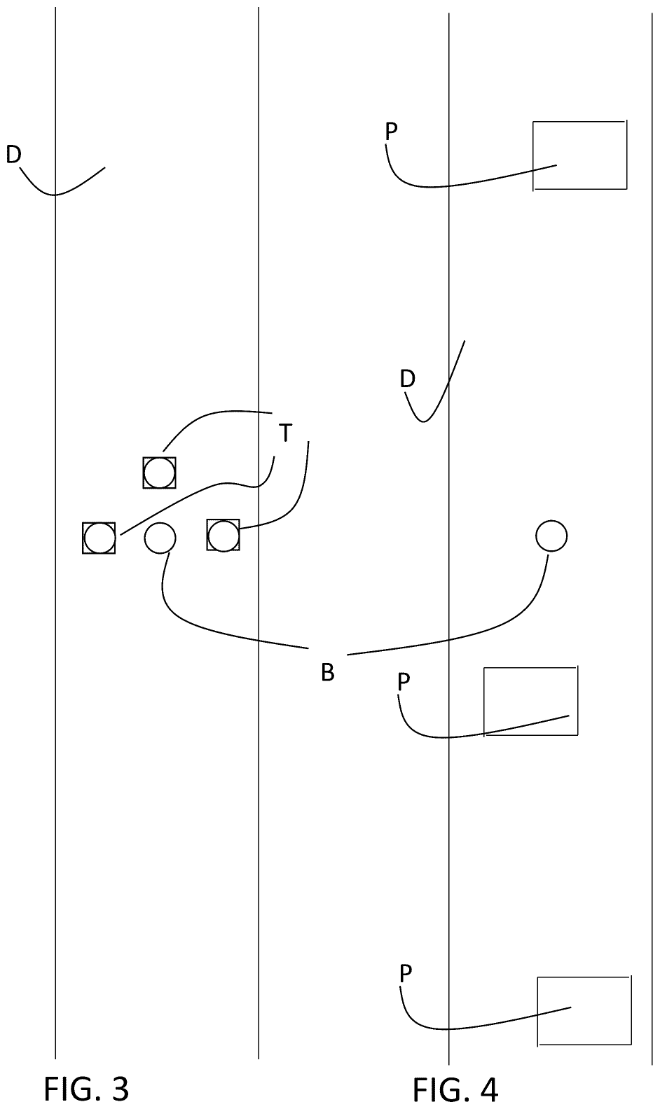

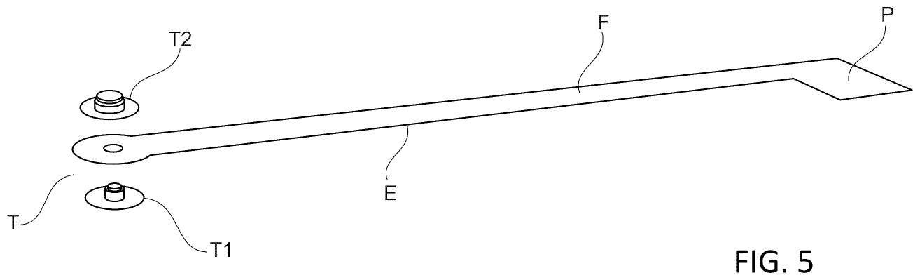

According to an embodiment, at least one of the terminals T is also a male or female element of a snap button. Furthermore, as illustrated in FIGS. 3 and 4, terminals T, unlike boss B, are separated from the skin thanks to first textile layer S1 so as not to touch the latter. Furthermore, terminals T are also protected by the adhesive film since the latter is arranged between terminals T and first textile layer S1. FIG. 5 shows an electrode unit E to which terminal T is connected e.g. by crimping, having a first and a second flange element T1, T2 rigidly connected one inside the other, first element T1 being of an electrically conductive material in contact with the electrically conductive ink and, therefore, in electrical continuity with pad P. The first and second flange elements T1, T2, when rigidly connected to each other, compress a corresponding portion of electrode unit E.

To complete the textile device, first layer S1 is glued onto second layer S2, in the embodiment of FIG. 2, the layers are folded lengthwise over each other to define a band D wearable around the chest (FIGS. 3 and 4). In this way, moreover, the adhesive sheaths the elongated filiform conductor F and further shields the electrical signal carried from disturbances, for example present due to the user's sweat. Therefore, an adhesive material, preferably a bi-adhesive film, more preferably the same bi-adhesive film described above is placed on both faces of elongated filiform conductor F. Preferably, the bonding of first layer S1, second layer S2 and electrode units E is achieved by hot pressing. Furthermore, in this configuration with a band height of 4 cm, applying a load of about 20 N results in 30% elongation and full elastic recover i.e. by 100%. It must be considered that, given the use of cardiac and respiratory monitoring for many hours, uses by children are also foreseen and, consequently, the height is purely indicative and must be suitable for the user.

A suitable closure device (not shown) adjusts the mutual position of corresponding portions of the ends of the band B so that the user can adapt the band itself to his own chest. The closure device can be made in various ways, both ‘open/closed’ and with continuous adjustment, for example through a zipper in which each side is fixed to the relative end portion: press-studs or traditional buttonholes applied to the end portions, a buttonhole carried by one end portion and cooperating with the other end portion in a sliding manner to be subsequently fixed by means of hook and loop, laces passing between the end portions and fixed either by means of a knot or by means of a sliding element with or without a spring commonly used to adjust in the circumferential direction anoraks and/or the tension of sports shoe laces etc. In general, the band is such that the first and second end portions face each other in the circumferential direction and approach each other when the closure device is tightened.

In particular, the closure device is manually adjustable by the user between a release configuration in which the band is not stretched and the wearable element is worn easily, and a tightening configuration in which band D is stretched in the circumferential direction to apply desired and sufficient pressure to the skin.

According to an alternative embodiment not illustrated band D is connected e.g. by stitching to a garment such as a top with straps, sleeveless shoulders or with sleeves in order to make it even more comfortable to wear for many hours e.g. the entire working day, as well as keeping band D firmly in place.

Finally, it is clear that modifications or variations can be applied to the textile device described and illustrated here without thereby departing from the scope of protection as defined by the attached claims.

For example, according to an embodiment not shown, terminals T and boss B are magnetic and preferably substantially flat. Consequently, the electronic control unit also comprises magnets e.g. permanent magnets, so the mechanical support of the electronic control unit on band B and the electrical data exchange connection between electrode units E and the electronic control unit are performed via a single magnetic device.

What is claimed is:

1. A band textile device comprising:
at least one electrode unit made by applying an electrically conductive polymer on a flexible sheet;
the electrode unit comprising a pad in contact with a skin of a user in use and an elongated filiform element silk-defining an electrical continuity with the pad;
the electrode unit being cut from sheet where the electrically conductive polymer is previously applied;
a first textile layer in use on a skin side having a first opening to allow the pad in use to contact the skin;
a second layer opposed to the first textile layer with respect to an electrode group and having a second opening to expose a portion of the filiform element;
at least one electrical terminal fixed in electrical continuity with the filiform element through the second opening and arranged on the surface of the second layer to be in use connected to an electronic control unit configured to receive and process an electrical signal of the pad;
a mechanical coupling device configured to carry the electronic control unit while being in electrical continuity with the electrical terminal;
at least one layer of adhesive material to attach the first textile layer to the second layer, the electrode group being interposed, to define a band and to sheath the elongated filiform element with adhesive; and
a closure device for releasably connecting a first and a second end portion of the band to each other and allowing in use the band to adhere to a skin of chest following adjustment.

2. The band textile device according to claim 1, wherein the pad is superimposed on the first textile layer passing through the first opening, an adhesive layer being present on the pad on an opposite side of the skin in use to attach the pad on the first textile layer.

3. The band textile device according to claim 2, wherein the pad is glued on the first textile layer out of the first opening along an elongation direction of the filiform element.

4. The band textile device according to claim 1, wherein ink is applied by screen-printing.

5. The band textile device according to claim 1, wherein a first thickness of ink on the pad is thinner or identical of a second thickness of the ink on the filiform element.

6. The band textile device according to claim 1, wherein the sheet is a fabric.

7. The band textile device according to claim 1, comprising a boss, configured in a metallic material, fixed to the first and/or second layer and having a first surface in use in contact with the skin and a second surface uncovered on the second layer in order to transmit a body heat of the user in use on the second surface.

8. The band textile device according to claim 7, wherein the second opening and the boss are close together wherein the electronic control unit, when fixed to the mechanical coupling device, is connected electrically and in heat exchange respectively with the terminal electric and with stud.

9. The band textile device according to claim 1, wherein the electrical terminal is in use separated from the skin by means of the first textile layer.

10. The band textile device according to claim 1, wherein the electrical terminals are configured to define further the mechanical coupling device.

11. The band textile device according to claim 10, wherein the electrical terminals and, where present, the boss define a non-axisymmetrical shape to define a single angular position to attach the electronic control unit.

12. The band textile device according to claim 7, wherein at least one of the terminal and the boss is made of a magnetic material.

13. The band textile device according to claim 1, wherein the sheet is added with ceramic particles to increase electrical insulation of applied ink.

14. A top comprising the band textile device according to claim 1.

15. A method of manufacturing the band textile device according to claim 1, comprising steps of:
applying ink on the sheet in an area larger than an area of the electrode unit;
and
cutting an electrode assembly and the ink from the area.

16. The method according to claim 15, comprising a step of applying a bi-adhesive layer on the sheet on an opposite side of screen printing before the step of cutting.

17. The band textile device according to claim 2, wherein ink is applied by screen-printing.

18. The band textile device according to claim 3, wherein ink is applied by screen-printing.

19. The band textile device according to claim 2, wherein a first thickness of ink on the pad is thinner or identical of a second thickness of the ink on the filiform element.

20. The band textile device according to claim 3, wherein a first thickness of ink on the pad is thinner or identical of a second thickness of the ink on the filiform element.
